(19) United States
(12) Plant Patent Application Publication
(10) Pub. No.: US 2004/0083523 P1
(43) Pub. Date: Apr. 29, 2004

(54) GYPSOPHILA NAMED 'DANGYPURNA'

(76) Inventor: Gabriel Danziger, Nir-Zvi (IL)

Correspondence Address: FOLEY AND LARDNER, SUITE 500, 3000 K STREET NW, WASHINGTON, DC, 20007, US

(21) Appl. No.: 10/339,345

(22) Filed: Jan. 10, 2003

(30) Foreign Application Priority Data
Oct. 24, 2002 (IL) 3407/02

(51) Int. Cl. A01H 5/00

(52) U.S. Cl. PLT/354

(57) ABSTRACT

A new and distinct Gypsophila plant named ‘Dangypurna’ characterized by having White semi-double flowers, globular shape, 8-10 mm corolla diameter; conic inflorescence form; 90-100 cm plant height, 50 - 60 cm plant width, erect, branching from plant base; suitable as a commercial product for cut flowers; and vigorous growth habit.

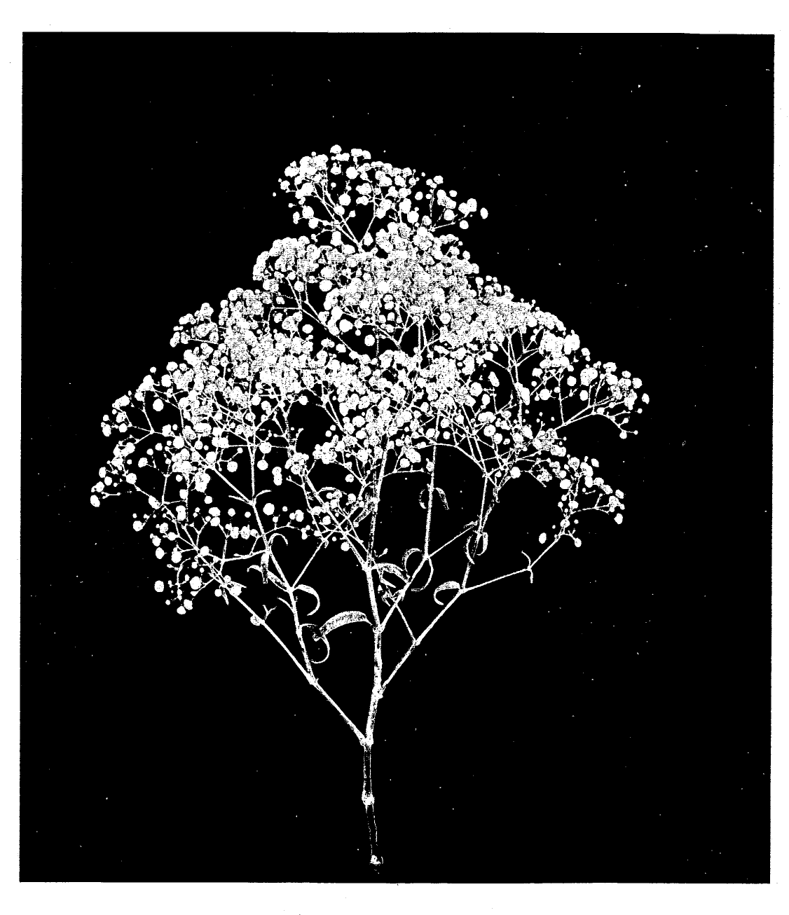

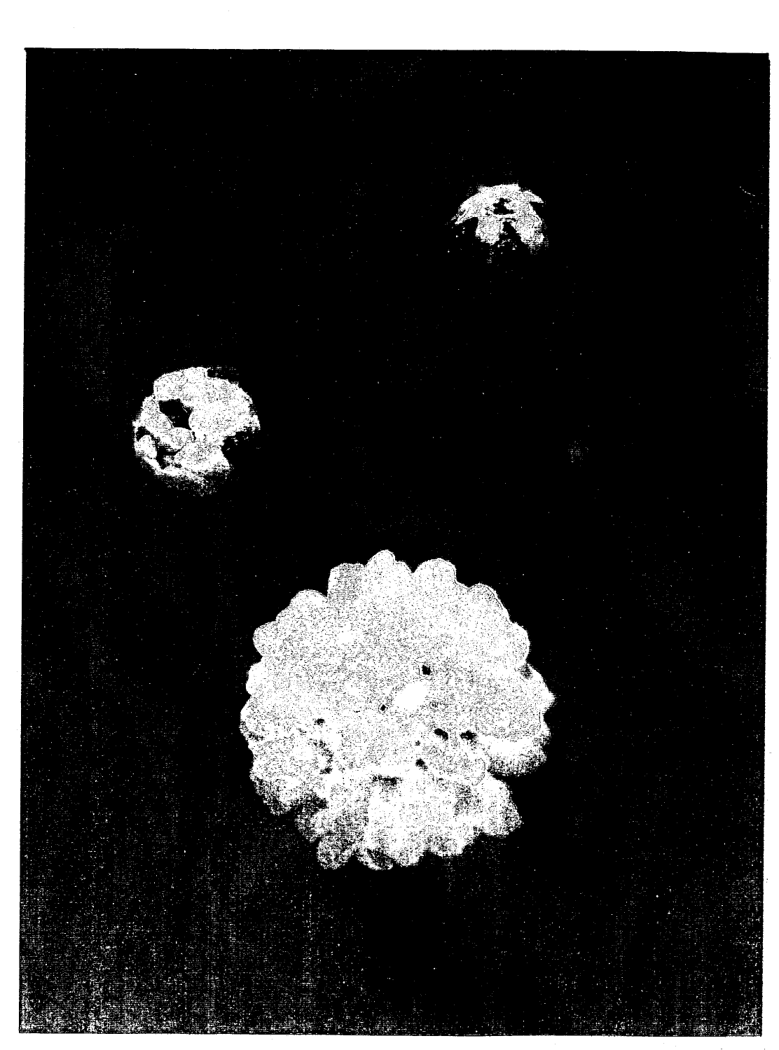

Latin name of the genus and species of the plant claimed:

Gypsophila paniculata

Variety denomination:

Dangypurna

### BACKGROUND OF THE INVENTION

The present invention relates to a new and distinct cultivar of Gypsophila plant, botanically known as Gypsophila paniculata, hereinafter referred to by the cultivar name ‘Dangypurna’.

The new cultivar originated from a cross made in a controlled breeding program in Mishmar Hashiva, Israel. The female parent is a proprietary unpatented cultivar from a hybrid line identified as ‘1102’. The male parent is open pollination. ‘Dangypurna’ was discovered and selected by the inventor, Gabriel Danziger, as a flowering plant within the progeny of the stated cross in a controlled environment in Mishmar Hashiva, Israel.

Asexual reproduction of the new cultivar by removing a cutting from the initial plant was first performed in December, 1999 in Mishmar Hashiva, Israel and has demonstrated that the combination of characteristics as herein disclosed for the new cultivar are firmly fixed and retained through successive generations of asexual reproduction. The new cultivar reproduces true to type.

### BRIEF DESCRIPTION OF THE INVENTION

The following traits have been repeatedly observed and are determined to be basic characteristics of ‘Dangypurna’ which in combination distinguish this Gypsophila as a new and distinct cultivar:

1. White semi-double flowers, globular shape, 8-10 mm corolla diameter;

2. Conic inflorescence form;

3. 90-100 cm plant height, 50-60 cm plant width, erect, branching from plant base;

4. Suitable as a commercial product for cut flowers; and

5. Vigorous growth habit.

‘Dangypurna’ has not been observed under all possible environmental conditions. The phenotype of the new cultivar may vary significantly with variations in environment such as temperature, light intensity, and daylength without any change in the genotype of the plant. The following observations, measurements and values describe the new cultivar as grown in Mishmar Hashiva, Israel under conditions which closely approximate those generally used in commercial practice.

Of the many commercial cultivars known to the present inventor, the most similar in comparison to ‘Dangypurna’ is the Gypsophila cultivar ‘Dangypflash’ (U.S. Plant Pat. No. 12,422). In comparison to ‘Dangypflash’, ‘Dangypurna’ has a higher stem yield, more upright growth habit and more symmetrical flowers.

### BRIEF DESCRIPTION OF THE DRAWING

The accompanying photographic drawings show typical flowers and foliage of a 12 week old ‘Dangyppink’ plant, with colors being as true as possible with illustrations of this type. The first drawing is a view of a flowering stem of ‘Dangypurna’. The second drawing is a a close-up view of the flower of ‘Dangypurna’.

### DETAILED BOTANICAL DESCRIPTION

The following observations, measurements and values describe the new cultivar at 12 weeks, as grown in Mishmar Hashiva, Israel under conditions which closely approximate those generally used in commercial practice. Regular cultural practices common for gypsophila in commercial fields were applied. Avoid excess GA or lighting. Color references are made to The Royal Horticultural Society Colour Chart (RHS), except where general colors of ordinary significance are used. Color values were taken under daylight conditions at approximately noon in Mishmar Hashiva, Israel.

Propagation: Stem cutting.

Plant:

General appearance and form.—Height: 90-100 cm. Width: 50-60 cm. Habit: Weak apical dominance, branching from plant base. Form: Upright bush type. Flowering response: Long day. Flowering season: Naturaly during summer; all year with suplemental lighting. Winter hardiness: Half hardy. Lastingness of the individual bloom: 10-12 days. Fragrance: Tyipical of gypsophila.

Foliage:

Amount.—Scarce.

Overall shape of leaf.—Lanceolate. Base: Truncate. Tip: Acuminate.

Margin.—Entire.

Texture.—Rough.

Main color of upper surface.—Mature leaf: Green 137-A. Immature leaf: Green 137-A.

Main color of lower surface.—Mature leaf: Green 137-B. Immature leaf: Green 137-B.

Venation.—None.

Size.—Length: 6-7 cm. Width: 1-1.5 cm.

Leaf base shape.—Truncate.

Leaf apex shape.—Acuminate.

Stipules.—None.

Inflorescence:

Natural flowering season.—Long day.

Corolla.—

Form.—Semi double to double depending on growing conditions.

Shape.—Globular.

Average number.—900-1000 flowers per flowering stem.

Size.—8-10 mm.

Petal number.—45-50.

Petal shape.—Spatulate with tip emarginated.

Petal markings.—None.

Petal color.—White RHS 155-D (both surfaces).

Stem:

Growth habit.—Upright and notably stable; the side stems are close to the main stem, forming inflorescence of several canopies, creating a wide and impressive structure; both flowers of the side stems and the main stem open almost simultaneously.

Average length.—90-100 cm.

Average diameter.—5-6 mm.

Color.—Yellow-Green 144-A.

Internode length.—3-4 cm.

Spur: None.

Bud:

Response.—Long day.

Color.—Greenish white.

Size before opening.—Length — 3 mm; width — 4 mm.

Pedicel Length.—8-10 mm.

I claim:

1. A new and distinct Gypsophila plant named ‘Dangypurna’, substantially as illustrated and described herein.
